(19) United States
(12) Patent Application Publication
(10) Pub. No.: US 2008/0058271 A1
(43) Pub. Date: Mar. 6, 2008

(54) Prevention of primary sjogren' s Syndrome by ICA69 deficiency

(76) Inventors: Hans-Michael Dosch, Toronto (CA); Shawn Winer, Toronto (CA)

Correspondence Address: MCHALE & SLAVIN, P.A., 2855 PGA BLVD, PALM BEACH GARDENS, FL, 33410, US

(21) Appl. No.: 11/895,892

(22) Filed: Aug. 28, 2007

Related U.S. Application Data
(63) Continuation of application No. 11/241,450, filed on Sep. 30, 2005, now abandoned.
Application No. 11/241,450 is a division of application No. 10/679,081, filed on Oct. 3, 2003, now abandoned.
(60) Provisional application No. 60/415,879, filed on Oct. 3, 2002.

(51) Int. Cl. A61K 38/00; A61P 37/00; G01N 33/564

(52) U.S. Cl. 514/14; 436/507

(57) ABSTRACT

This invention relates to identification of an autoantigen implicated in the development and progression of Sjögren's Syndrome (pSS); particularly to the disease modifying effect of creating a deficiency in the ICA69 autoantigen; and most particularly to development of diagnostic and therapeutic avenues, means for the differential diagnosis of pSS versus other autoimmune disease, e.g. Systemic lupus erythematosis (SLE), and procedures for immunotherapeutic treatment effective to alter the course and progression of pSS.

### CROSS-REFERENCE TO RELATED APPLICATIONS

This application is a continuation of U.S. patent application Ser. No. 11/241,450, filed Sep. 30, 2005, which is a divisional application of U.S. patent application Ser. No. 10/679,081, filed on Oct. 3, 2003, which relies upon U.S. Provisional Patent Application No. 60/415,879, filed Oct. 3, 2002, the contents of both are herein incorporated by reference in their entirety.

### FIELD OF THE INVENTION

This invention relates to identification of an autoantigen implicated in the development and progression of primary Sjögren's Syndrome (pSS); particularly to the disease modifying effect of creating a deficiency in the ICA69 autoantigen; and most particularly to development of diagnostic and therapeutic avenues, means for the differential diagnosis of pSS versus other autoimmune disease, e.g. Systemic lupus erythematosis (SLE), and procedures for immunotherapeutic treatment effective to alter the course and progression of pSS.

### BACKGROUND OF THE INVENTION

Primary Sjögren's Syndrome (pSS) is a common, chronic autoimmune disorder of unknown etiology, affecting exocrine glands, primarily (90%) in middle-aged women with a prevalence varying between 0.3-4.8%, depending on region and diagnostic criteria.

Despite considerable efforts to find evidence of an initiating viral trigger, the cause of Sjögren's Syndrome remains unknown. The disease leads to lacrimal and salivary dysfunction, with dryness of mouth and eyes leading to considerable surface damage and attendant chronic discomfort and pain. The disease involves activation of CD4-predominant T cells and of B lymphocytes with autoantibodies detectable in the circulation, and associated with complications such as vasculitis and interstitial pneumonitis. The chronic B cell activation can lead to the slow emergence of autonomous clones of B cells that can evolve into non-Hodgkin's lymphoma at a rate that is 44 times that of the general population (an incidence around 6.5%). There is growing evidence that a subset of patients may have or develop multiple sclerosis. Liver disease such as Primary Biliary Cirrhosis and Autoimmune Hepatitis can be associated with Sjögren's Syndrome.

Pathologically, the hallmark of pSS is a CD4-predominant glandular T cell infiltrate that is initially periductal, and later leads to B cell and plasma cell accumulation. The secretory defect occurs disproportionately to the degree of acinar destruction, such that the early dryness is thought to result from immunological targeting of the muscarinic 3 parasympathetic receptors within the glands. Infiltrates in salivary and/or lacrimal glands, eventually lead to tissue destruction, and this is thought to occur in part because of targeting of a number of autoantigens, such as alpha and beta fodrin, and protein fragments associated with intracellular RNA, such as Ro and La. The original observation of the instant inventors of strong protection from salivary, and complete absence of lacrimal disease in ICA69-deficient NOD mice was unexpected, as previous work associated this autoantigen specifically with human and NOD type 1 diabetes, and, more recently, multiple sclerosis. ICA69 is a self-antigen expressed in brain, pancreas, salivary and lacrimal glands. NOD-strain mice represent a premier animal model of spontaneous pSS.

Organ-selective autoimmune disorders are characterized by broad spreading to multiple target autoantigens, and the genetic removal of any one such antigen was expectedly not associated with significant disease impact in autoantigen gene knockouts (GAD65, ICA69, IA2), the recent observation of T1D protection in insulin-1 knockouts raised questions of the degree of backcrossing, since heterozygous animals also show protection. Reduced antigen spreading may set Sjögren's Syndrome apart, perhaps due to lesser involvement of CD8+ T cells that drive disease progression in conditions such as autoimmune diabetes.

The clinical picture varies and can be stable or progressive, occasionally leading to life threatening complications. Therapeutic approaches in pSS are symptomatic and, on the whole, considered inadequate. It is often difficult to justify the routine use of immunosuppressive drugs because the disease is so localized, and the downside of these medications would seem to be excessive, in particular considering the possible risk of accelerating lymphoma and increased risk of infection. As in other autoimmune disorders, most immunosuppressants tested have shown limited effectiveness in Sjögren's Syndrome. Thus pSS is a prototypical, tissue-selective autoimmune disorder, and it shares many fundamental aspects with its cousins, MS, type 1 diabetes, Crohn's disease and others.

Animals can develop homologs of Sjögren's Syndrome. The premier pSS model, NOD-strain mice, provide the closest approximation of the human disease. NOD pSS develops independently of type 1 diabetes, and does not require the diabetes-prerequisite NOD MHC class II (I-Ag7). We have generated knockout mice, deficient in the diabetes autoantigen, ICA69, and bred the null allele onto NOD congenic animals. While Type 1 diabetes (T1D) development proceeded at slower rate but normal incidence, these mice showed a dramatic reduction of pSS, with complete prevention of the lacrimal disease typical for old males.

In wild-type NOD mice, immunotherapeutic induction of tolerance to ICA69 has been optimized and is effective at reversing sialoadenitis and dacryadenitis even in late stage disease.

Autoimmunity in, for example, Type 1 diabetes, is characterized by progressive spreading to many different autoantigens, and to more epitopes within each. The inability of ICA69 deficiency (or for that matter, GAD65 or IA2 deficiency) to affect T1D outcome was therefore not surprising. This, then, sets pSS apart, and suggests that autoimmunity in this disease is considerably more narrow with less antigen spreading, perhaps consistent with the surprising effectiveness of ABBOS immunotherapy. pSS protection was complete only for lacrimal disease, but there was low grade, and less progressive salivary disease in the KO mice, suggesting that the process underlying and driving the autoimmune attack was still at work, presumably targeting otherwise perhaps minor target autoantigens.

Initially, ABBOS mediated pSS protection was not quite uniform, and a subset of treated animals showed little protection, a few even disease acceleration. This was not surprising, and likely dose related, since previous work had demonstrated that a suboptimal ABBOS dose can mimic the effect of Tep69 and precipitate disease. These observations were initially made in animals receiving single injections, however treatment protocols have now been optimized, and the instantly disclosed protocol shows no acceleration.

In a small study of pSS patients, nearly all had prominent T cell autoreactivity to ICA69, that targeted the same epitope as the immunodominant target typical for T1D.

As a necessary prelude to phase I immunotherapy trials, it is now proposed to use NOD mice to further optimize pSS immunotherapy for subsequent translation to the human system, extend studies of ICA69 autoimmunity in pSS patients (and their relatives), establish MHC immunogenetics of these T cell responses, systematically map human pSS epitopes and conduct T cell mechanistic studies.

These studies are expected to form a rational basis for tolerance-inducing peptide infusions alone or in combination with other disease modifying drugs in pSS patients. Since the Syndrome is largely localized to salivary and lacrimal glands, direct tissue access and secretory function measures are possible, and indeed have been used to assist in the routine diagnosis of pSS. This disease thus appears to be a prime candidate to become the test- and development platform for immunotherapy of organ-selective autoimmune diseases in general, which has so far failed to translate broadly encouraging rodent data to humans.

Glossary of Terms:

ABBOS T cell epitope in bovine serum albumin (BSA).

IFA incomplete Freund's adjuvant (water-oil emulsion).

MHC major histocompatibility complex, e.g. HLA in humans, H-2 in mice.

Mimicry antigenic cross-reactivity: e.g. Tep69 & ABBOS peptides are recognized by the same T cell clones and auto-antibodies.

NOD non-obese diabetic mice, develop primary Sjögren's Syndrome spontaneously and independently of Type 1 diabetes.

Tep69 T cell self-epitope in ICA69.

### DESCRIPTION OF THE PRIOR ART

U.S. Pat. No. 6,207,389 is directed toward methods of controlling T lymphocyte mediated immune responses and to methods of detecting subjects at risk for developing Type I Diabetes by detection of antibodies to p69 protein.

### SUMMARY OF THE INVENTION

In accordance with the present invention the genomic ICA69 locus was inactivated, thereby generating ICA69-deficient NOD congenic mice which were subsequently analyzed for the development of pSS. ICA69 autoimmunity was analyzed in controls or patients with primary SS or SLE, and in various NOD mice, some treated with an ICA69-directed prototype peptide vaccine.

Disruption of the ICA69 locus was found to prevent lacrimal and dramatically reduced salivary gland disease in NOD mice. In normal NOD mice, ICA69-specific T-cells accumulated in lymph nodes draining salivary tissue. Patients with primary SS, but not SLE patients, nor healthy control subjects, had similar T- and B-cell autoreactivity against ICA69. Immunotherapy with a high-affinity mimicry-peptide targeting ICA69-specific T-cells produced long-term reduction of established pSS in wild type NOD mice.

ICA69 is a new autoantigen in primary SS that plays a critical role in disease progression and may be of diagnostic value. Immunotherapy of primary SS with a high-affinity mimicry-peptide targeting ICA69-specific T-cells appears to be promising, since autoimmunity in NOD pSS appears uniquely susceptible to such treatment even late in disease.

Accordingly, it is an objective of the instant invention to identify an autoantigen implicated in the development and progression of Sjögren's Syndrome (pSS).

It is a further objective of the instant invention to demonstrate the disease modifying effect of creating a deficiency in the ICA69 autoantigen.

It is yet another objective of the instant invention to develop diagnostic and therapeutic avenues for treatment of pSS.

It is a still further objective of the invention to provide means, e.g. diagnostic assays, for the differential diagnosis of pSS versus other autoimmune disease, e.g. Systemic Lupus Erythematosis (SLE).

It is yet an additional objective of the invention to develop procedures for immunotherapeutic treatment effective to alter the course and progression of pSS.

It is a still further objective of the instant invention to teach a transgenic animal, particularly an ICA69 deficient NOD mouse, which essentially does not develop pSS.

Other objects and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE FIGURES

The instant patent or application file contains at least one drawing executed in color. Copies of the patent or patent application publication with color drawing(s) will be provided by the Office upon request and payment of the necessary fee.

FIG. 1. Protection from sialoadenitis and absence of dacryoadenitis in ICA69 deficient NOD mice.

FIG. 2. Measurement of T cell proliferative responses to ICA69, its dominant epitope, Tep69, BSA, and its dominant NOD mouse epitope, ABBOS, measured in lymph nodes draining the pancreas.

FIG. 3. Modification of sialoadenitis by peptide-based immunotherapy.

FIG. 4. Splenic T cell responses to Tep69 in ABBOS-treated mice with persistent sialoadenitis (n=6, green or blue shading in A), and mice with peptide-mediated disease reduction (n=11, red shading in A).

FIG. 5. Pilot studies were used to hone in on 3 variables: peptide dose, route of administration (i.v., i.p., s.c.) which effect the success of pSS immunotherapy.

FIG. 6. Illustration of effectiveness of ABBOS peptide-based vaccine, and involvement of anti-mACHR autoantibodies in affecting salivation.

FIG. 7. T and B cell autoimmunity to ICA69 in patients with primary SS, and SLE versus healthy controls.

FIG. 8. T cell and B cell autoimmunity to ICA69 in patients with pSS.

### DETAILED DESCRIPTION OF THE INVENTION

Discussion of Figures

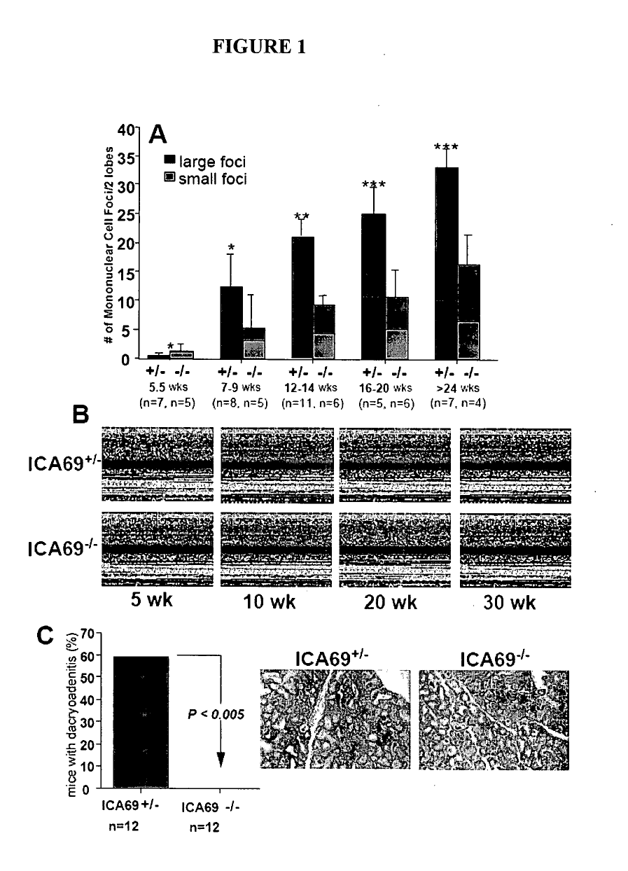

FIG. 1. Protection from sialoadenitis and absence of dacryoadenitis in ICA69 deficient NOD mice. (A) Female ICA69^{±} and ICA69^{-/-} NOD mice were sacrificed at various ages and the number of mononuclear cell foci in both submandibular glands were enumerated. *P>0.1; **P<0.01; ***P<0.001. (B) Representative histopathology of submandibular glands from ICA69^{±} and ICA69^{-/-} NOD mice of various ages (H&E stains, 40× magnification). (C) Histological signs of dacryoadenitis, observed in most ICA69^{±} NOD males, is absent in ICA69^{-/-} NOD males aged 35-40 weeks (H&E stains, 100× magnification).

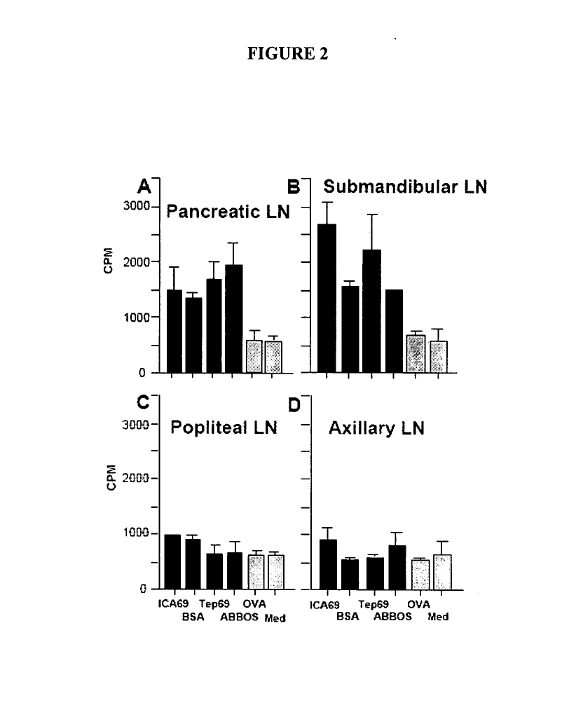

FIG. 2. T cell proliferative responses to ICA69, its dominant epitope, Tep69, BSA, and its dominant NOD mouse epitope, ABBOS, were measured in lymph nodes draining the pancreas (A), and submandibular glands (B), or lymph nodes draining the lower (C) or upper (D) extremities. Gray columns: control cultures stimulated with ovalbumin (OVA) or Medium (MED) only. To obtain sufficient cell numbers, lymph node cells were pooled from seven mice. One of three similar data sets is shown.

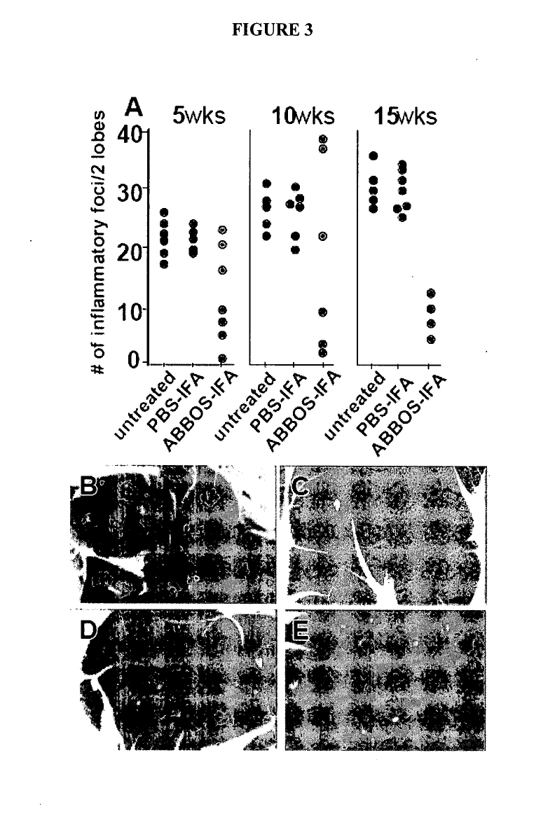

FIG. 3. Modification of sialoadenitis by peptide-based immunotherapy. (A) 10 week-old NOD females received 200 ug ABBOS i.p. in incomplete Friend's adjuvant (IFA), vehicle only (PBS) or were left untreated. Sialoadenitis scores were measured 5, 10 or 15 weeks later. Colour key: protected mice—red, unchanged sialitis—green, enhanced disease—blue. (B) Submandibular gland from a 20 wk old NOD female previously injected with PBS-IFA. Absence (C), reduction (D), or increase (E), of sialoadenitis in submandibular glands from 20 wk old NOD females injected with ABBOS peptide 10 weeks earlier (H&E stains, 40× magnification).

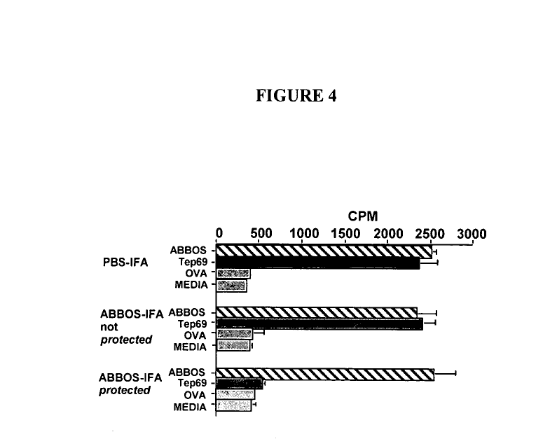

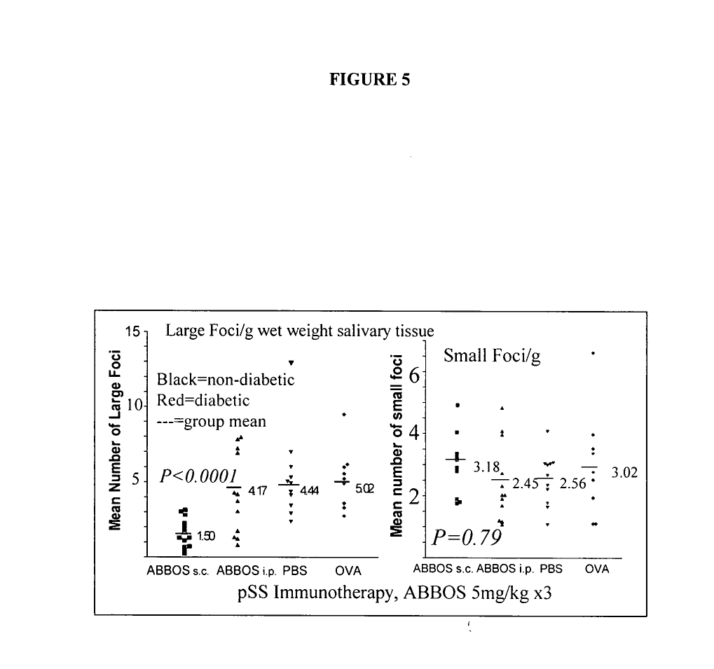

FIG. 4. Mechanisms of immunotherapy-induced disease protection are at best partially resolved in general. In terms of T cell autoreactivity in the present context, only protected animals showed an absence (fat arrow vs. thin arrow) of T cell pools that recognized both, ABBOS and its endogenous ICA69 mimicry peptide, Tep69 (see figure). The instant inventors have constructed ICA69 transgenic NOD mice which showed deviation of mimicry T cells recognizing the Tep69 epitope as well as ABBOS: these mice were protected from autoimmune disease, and formally demonstrated the protective abilities of ABBOS-only T cell pools noted earlier in functional studies in NOD mice and humans. The explanation for these observations is, almost certainly, that deviation of the fine specificity of T cell receptors for Tep69 is associated with loss of pathogenicity in the remaining T cell pools. However, it remains possible that lasting T cell anergy might play a role in disease protection and/or the undetectability of relevant (Tep/ABBOS-specific) T cell pools. FIG. 5. Pilot studies were used to hone in on 3 variables: peptide dose, route of administration (i.v., i.p., s.c.) and injection schedules (the published data (Lancet) used single injection, 100 μg, s.c. in IFA (i.e. oil), the latter would not likely be usable in humans and we now have tested i.p. without IFA). We also refined our sialitis scoring system to include a correction for gland weight: this strategy reduced variability even of previous data considerably, enhancing our statistical power. To our surprise, i.v. injection (effective at T1D prevention) failed to affect pSS. Pilot studies also suggested that 3 injections 2 weeks apart were more effective than a single large injection, and this schedule prevented unsuccessful as well as accelerating outcomes.

The new data shown in the figure derive from a large, complete experiment to test these pilot suggestions. “Large infiltration foci” are the main pathogenic infiltrates associated with tissue destruction and disease progression. Injection of ABBOS, 5 mg/kg (roughly equivalent to 100 μg/mouse) turned out to be an effective dose, 3-10 times larger doses were not more effective. We chose to begin therapy at an age of 10-12 weeks, when salivary disease in females is well established with high incidence (around 85% in our colony and at least mild disease in most animals). Two further injections followed, 3 weeks apart. As shown in the figure, s.c. injection was the superior route (p<0.0001 vs. ABBOS i.p., PBS or OVA peptide injection). Salivary data were obtained in females at 25-30 weeks or earlier if animals developed T1D (red symbols).

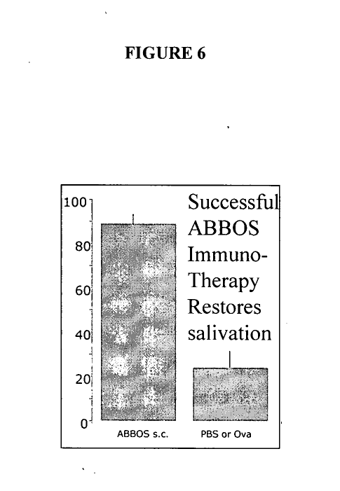

FIG. 6. The plausible involvement of anti-mACHR autoantibodies in affecting salivation, requires consideration. The ABBOS peptide-based ‘vaccine’ was effective in NOD pSS, as judged by pathohistology (scoring of infiltrative foci) and data on recovery of secretory function (Figure, p=0.002 ABBOS vs. control treatments). However, no data were obtained on mACHR autoantibodies. Given the rather short Ig-half life times in mice, and the almost certain T helper cell dependency of such antibodies, it is possible that T cell directed immunotherapy will reduce autoantibody levels, and secretory function. Normalized exocrine secretion may imply that successful immunotherapy does affect autoantibodies that interfere with secretion.

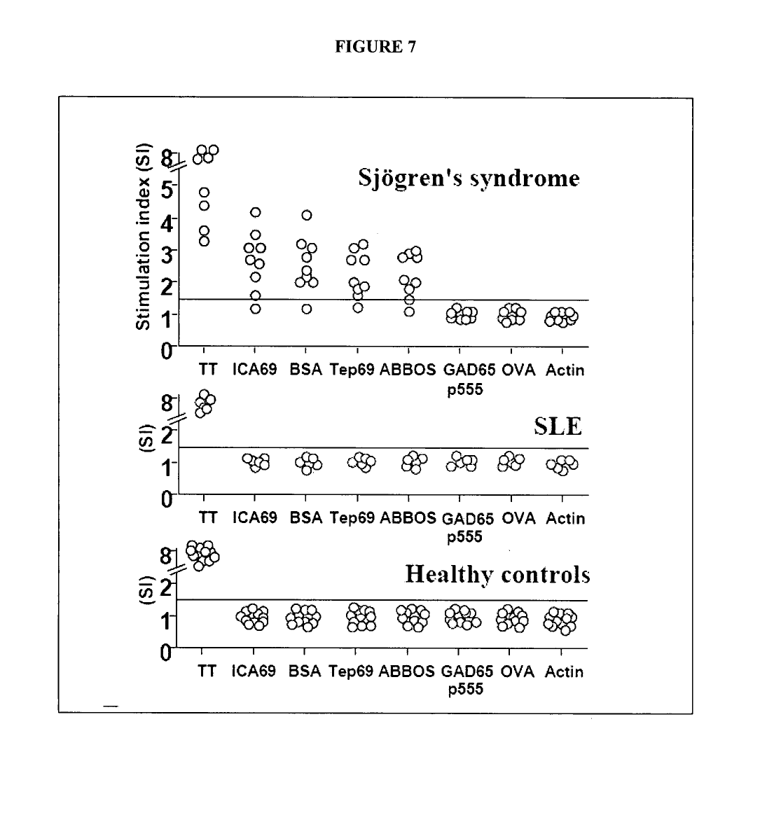

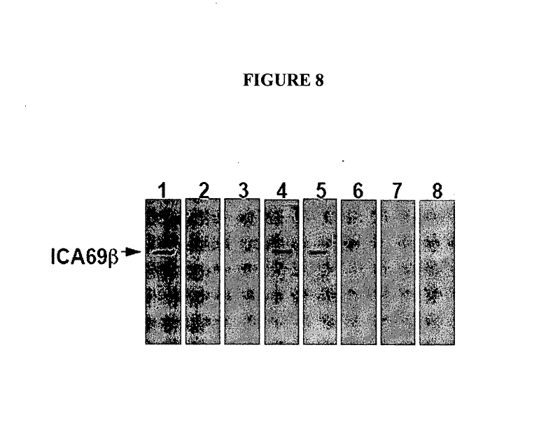

FIG. 7.(A) T cell responses to ICA69, BSA, Tep69, and ABBOS were analyzed in patients with primary SS (n=9) SLE patients (n=6) or healthy controls (n=12). Positive responses to tetanus toxoid (TT) contrasted with negative responses to OVA, actin or the type 1 diabetes-associated GAD65 peptide, p555. Data are expressed as stimulation index (SI, experimental/background cpm, as described herein). Background counts were similar in all cohorts (mean±SD: 1154±354 cpm). FIG. 8. Autoantibodies to ICA69 (1 μg protein/lane) were detected in Western blots of sera (1 to 1000 dilution) from patients with pSS (lanes 1-5) but not in controls (lanes 6-8).

pSS is a chronic autoimmune disease characterized by lymphocytic infiltration and destruction of exocrine glands, in particular in salivary and lacrimal tissue¹. Destruction of these glands often results in dryness of the eyes (keratoconjunctivitis sicca), and mouth (xerostomia). The prevalence of the disease is high, with about 1% of the population affected, most being females. Both organ selective and systemic autoimmunity are thought to participate in disease progression. As with other organ-selective autoimmune disorders, there is evidence for multiple environmental and genetic factors that contribute to disease risk in pSS^{2,3}. Several candidate autoantigens associated with pSS have been identified and some are currently used in disease diagnosis. Of these, SS-A/Ro, SS-B/La, and the recently identified SS-56 are considered systemic autoantigens and have been linked to other autoimmune diseases such as systemic lupus erythematosus (SLE)^{4,5}. In addition, autoantigens such as a-fodrin, b-fodrin, and the muscarinic M3 receptor are considered tissue-restricted autoantigens in pSS^{6,8}. The pathogenic roles of these autoantigens in the initiation and progression of pSS are unclear, but antibodies against the muscarinic M3 receptor may participate in the loss of salivary function⁸. pSS treatment is essentially symptomatic. Identification of new autoantigens and their pathogenic roles could have considerable impact on design of new diagnostic and therapeutic strategies¹.

Several animal models have been used to study pSS, including the nonobese diabetic (NOD) mouse, the MRL/lpr mouse and the NFS/sld mouse, thymectomized 3 days after birth⁹⁻¹¹. Among these, the NOD mouse may represent the premier model, since, like in human pSS, loss of salivary secretory function develops spontaneously^{8,12}. The NOD mouse is also the premier model for spontaneous type 1 diabetes, but the two diseases can be separated genetically; for example, NOD.H-2^{b} mice develop pSS, but not diabetes¹³. NOD mice, like human diabetes patients and many relatives with a high genetic risk to develop diabetes, lose tolerance to the islet cell autoantigen 69 kDa, ICA69^{14,15}. ICA69 is a conserved protein of unknown function whose expression pattern includes neurons, pancreatic b-cells, salivary and lacrimal glands¹⁶⁻¹⁸. T- and B lymphocytes from NOD mice and the majority of diabetes patients target primarily the ICA69-36 epitope, Tep69, although other cryptic epitopes likely exist^{14,15}. ICA69 (but not its Tep69 epitope) is also targeted in multiple sclerosis¹⁹. We recently generated speed-congenic ICA69-deficient NOD mice to analyze the role of ICA69 in autoimmunity¹⁷. These animals develop Type 1 diabetes with slight delay at essentially wild type rates, assigning a facultative rather than obligate role to ICA69 in diabetes development¹⁷.

In accordance with the instant invention, it has been determined that ICA69 deficient NOD females have dramatically impaired development of pSS and its associated exocrinopathy. Modification of T cell immunity to ICA69/Tep69 by immunotherapy prevented disease development and reduced established disease in wild type NOD mice. Extending these observations to humans, we observed both T cell and autoantibody responses to ICA69 in pSS patients, but not in healthy controls or patients with SLE. The instantly disclosed data establish ICA69 as a new pSS autoantigen which appears to be critically involved in disease progression.

Methodology

Human Subjects

Blood samples were obtained from patients (n=15) with primary SS or SLE at the Arthritis Center at Toronto Western Hospital and from healthy, adult volunteers through ethics-board approved consent (n=12). pSS patients were female, had documented xerostomia and xerophthalmia and met San Diego disease criteria. All were anti-Ro antibody positive, 6 had anti-fodrin autoantibodies and all had minor salivary gland biopsy focus scores of >5. Healthy controls (n=12) of similar age and gender profile were recruited from staff. Fresh blood was used for T cell studies. In immunoblotting experiments, sera from patients and controls were diluted at 1:1000 and blotted on nitrocellulose containing 1 mg of recombinant ICA69 protein to detect the presence of anti-ICA69 antibodies.

Mice.

NOD/Lt (H2-IA^{g7}.) mice were bred and maintained according to approved protocols in our conventional unit (85% diabetes incidence in females, 36 weeks of age). This study was based on experiments with approximately 200 mice. The generation of ICA69^{-/-} speed congenic NOD mice has been described ⁷. In these animals, all 17 Idd loci²⁰ were homozygous NOD as assessed with microsatellite markers in the 5^{th} backcross generation¹⁷. Knockout animals in this report were derived from the 10^{th} backcross.

Mouse Histology.

Submandibular and lacrimal glands were removed and fixed in 10% buffered formalin for at least 24 hr. Tissue sections were stained with hematoxylin/eosin. For sialoadenitis scoring, two blinded observers enumerated the number of mononuclear foci at 3-5 different tissue depressions (100 mm/depression) in 2 full glands from each animal. The scores from the different levels and the two observers were averaged. A ‘small’ mononuclear focus had <75 inflammatory cells/section (400× magnification). A large focus had >75 inflammatory cells. Dacryoadenitis was diagnosed if at least one mononuclear focus was detected in one of two lacrimal glands from each mouse. In ICA69^{±} and wild type NOD mice, dacryoadenitis often consisted of large masses of lymphocytes infiltrating into the acinar tissue. Such infiltrations were absent in all ICA69^{-/-} animals analyzed.

Proteins, Peptides and Immunotherapy.

Human recombinant ICA69-b was purified as described¹⁴. Grade V bovine serum albumin (BSA) and Ovalbumin (OVA) were purchased (Sigma, St. Louis, Mo.). Peptides were purchased HPLC purified (>95%) and confirmed by mass spectroscopy (numbers indicate the N-terminal amino acid position): Tep69 (ICA69-p36), AFIKATGKKEDE; ABBOS (BSA-p150), FKADEKKFWGKYLYE. In immunotherapy experiments, NOD female mice, 10 weeks of age, were given a single intraperitoneal injection (100 ml) of either 200 mg ABBOS peptide or PBS, both emulsified at a 1:1 ratio in incomplete Freund's adjuvant (IFA). Control mice were untreated. Organs were harvested for histopathology at various times after treatment.

Proliferative T cell Responses.

NOD lymph-node, spleen and human peripheral blood T cell responses were measured with three slightly different protocols. Draining lymph node cells from 10 week old NOD females were pooled. 2×10⁵ lymph node cells along with 2×10⁵ irradiated (1100 rad) syngeneic spleen cells were cultured in serum-free AIM-V media (Life Technologies, Mississauga, Ontario, Canada) in the presence of protein or peptide antigen. Proteins (ICA69, BSA or OVA) were used at concentrations of 5 mg/ml, peptides (Tep69, ABBOS) at 50-100 mg/ml²¹. After 72 hr of incubation, cultures were pulsed overnight with 1 mCi of [³H]thymidine, harvested and subjected to liquid scintillation counting. Experiments were repeated three times with similar results, each with lymph nodes pooled from groups of 4-7 mice. Proliferation assays with spleen cells used 4×10⁵ responding cells/well and no irradiated splenocytes. For the detection of human T cell responses, Ficoll-Hypaque purified peripheral blood mononuclear cells (PBMC) were cultured at 10⁵ cells/well for one week in serum-free Hybrimax 2897 medium (Sigma) supplemented with human IL-2 (10 U/well) and 0.01-10 mg of antigen¹⁵. This assay performed well in a large, blinded study and in the first international T cell workshop of the Immunology of Diabetes Society²².

Statistics.

Proliferative T cell responses were expressed as stimulation index (SI, experimental/control cpm). SI's greater than the mean SI in OVA-stimulated cultures plus 3 SD were deemed positive¹⁵. Numeric data were compared by Mann-Whitney tests, Fisher's exact test was used to analyze tables. All P values were two-tailed and significance was set at 5%. Figures present mean values plus lSD.

Protection from pSS in ICA69 Deficient NOD Congenic Mice.

The expression of ICA69 is similar in humans and rodents¹⁸ and its presence in the submandibular glands of NOD mice¹⁷ led us to examine the impact of ICA69 deficiency on the development of NOD mouse sialoadenitis and dacryoadenitis. Submandibular glands from NOD, ICA69^{±} and ICA69^{-/-} NOD females of various ages were analyzed by two blinded observers for the number and size of mononuclear cell infiltration foci, values were within ±10%. Number and size of mononuclear cell foci increased progressively with age in heterozygous (ICA69^{±}) mice (FIGS. 1A, B top panel). Timing and progression of sialoadenitis in ICA69^{±} and wild type mice was similar (P values >0.20, data not shown, equivalent to FIG. 3A “untreated”), with initial infiltrates observed by 5-7 weeks of age. In striking contrast, sialoadenitis was significantly reduced in ICA69^{-/-} NOD mice (FIGS. 1A, B bottom panel). Beginning usually around 9-10 weeks of age, ICA69 deficient animals developed mild salivary gland infiltrations, that showed slow progression, on average 55-65% below submandibular gland mononuclear foci observed in wild type or heterozygote mice (P 0.006 vs. ICA69^{±} mice). While ICA69 is not absolutely required for disease initiation, its absence plays a lasting role during expansion of the disease process, which shows little progression in females older than 6 months of age. ICA69 therefore appears to be involved in the progression of disease.

pSS in male NOD mice differs from the female phenotype, with less sialoadenitis, but pronounced dacryoadenitis²³. The cause of this gender bias is unclear, but unequal salivary and lacrimal gland disease is common also in human pSS. Small perivascular and periductal lymphocytic infiltrates of the NOD male lacrimal gland appear around 10 weeks of age. By 30-40 weeks of age, dacryoadenitis is conspicuous with extensive lymphocyte infiltration into the acinar tissue and progressive tissue destruction. In our colony, about two thirds of wild type and 7/12 ICA69^{±} NOD males between the ages of 35-40 weeks exhibit definitive dacryoadenitis (FIG. 1C). In similarly aged male ICA69^{-/-} NOD mice, dacryoadenitis was undetectable (0/12, FIG. 1C). Spontaneous autoimmune inflammation of the lacrimal gland appears to require ICA69 expression.

ICA69-Specific T Cell Autoreactivity in the NOD Mouse.

These observations suggested a key role for ICA69 expression in the development and progression of NOD mouse pSS. This phenotype could reflect a role for ICA69 as an autoantigen or a role for ICA69 protein-function. To begin an analysis of these two alternatives, we measured T cell autoreactivity to ICA69 and its immunodominant T cell epitope, Tep69, in 10 week old NOD females. Proliferative in vitro recall responses were assessed in draining lymph nodes from various tissues, in order to localize where T cell tolerance to ICA69 was lost. Proliferative T cell responses to ICA69 and Tep69 were detected in both pancreatic and submandibular lymph node cells (FIGS. 2A, B), but not in popliteal or axillary lymph nodes (FIGS. 2C, D). Equally exclusive to pancreatic and submandibular lymph node cells, we observed T cell proliferative responses to bovine serum albumin (BSA) and its immunodominant epitope ABBOS, a peptide that displays amino acid homology and antigenic mimicry with Tep69¹⁴. Spleen cell responses to ICA69, Tep69, BSA and ABBOS were present as previously described by us²¹ and others²⁴ (data not shown, but for example see FIG. 3F). The localization of spontaneous ICA69 immune responsiveness to the submandibular lymph nodes specifically links ICA69 autoimmunity with the salivary glands, and suggests that ICA69 is a candidate autoantigen in NOD mouse pSS.

To test this conclusion and determine the role for ICA69 autoimmunity in the progression of NOD mouse pSS, we employed an immunotherapy strategy¹⁴. Treatment of NOD mice with the ABBOS mimicry peptide induces long lasting T cell tolerance to Tep69 in most animals, due to the high MHC class II affinity of ABBOS²¹. We examined the effects of ABBOS peptide-induced Tep69-specific T cell tolerance on the development and course of NOD mouse sialoadenitis. In order to detect possible therapeutic effects of the peptide, we injected 10 wk old wild type NOD females with established disease. Five, 10 and 15 weeks after a single intraperitoneal injection of 200 mg ABBOS emulsified in oil (incomplete Freund's adjuvant, IFA), submandibular glands were examined for the number of mononuclear foci (FIG. 3A). Control mice, untreated or injected with emulsified vehicle only, showed severe and progressive sialoadenitis at all time intervals after treatment (FIGS. 3A, B). ABBOS treatment produced variable results, with predominant disease protection in two thirds of animals (P<0.001, FIG. 3A red circles, C, D). In a third of protected mice, sialoadenitis was reduced to nearly absent (FIGS. 3C, D). However, in contrast to disease protection, we observed moderate disease exacerbation in a subset of ABBOS treated mice (2/17 mice analyzed (12%), FIG. 3A blue circles, E). Thus, a single injection of the immunotherapeutic agent, ABBOS²¹, can affect progression and induce regression of established NOD Sjögren's disease. To analyze the variability of disease effects observed following ABBOS-immunotherapy, we measured relevant splenic T cell autoreactivity 5, 10 and 15 weeks following treatment and compared the outcome with disease status. As expected^{14,21}, mice treated with emulsified buffer (PBS-IFA, n=6) had T cell recall responses to both, Tep69 and ABBOS peptides (FIG. 3F). Similarly, we observed Tep69 and ABBOS proliferative responses in ABBOS treated animals that were not protected from disease (FIG. 3F, n=6), including mice that displayed moderate disease exacerbation. However, T cell responses to Tep69 were greatly reduced in those mice that displayed protection from sialoadenitis (n=11). Thus, ABBOS treatment had selectively eliminated mimicry T cell pools that could recognize the self-peptide, Tep69, inducing a bias for ABBOS recognition only. The presence of ABBOS, but not Tep69 T cell responses following ABBOS immunotherapy of the NOD mouse was previously associated with diabetes prevention¹⁴, and likely reflects selection of lower affinity T cell pools that cannot be activated by Tep69 due to its very low MHC class II affinity²¹. Taken together, these data indicate that ICA69/Tep69 specific T cell pools are critical in sustaining the natural progression of sialoadenitis in NOD mice, and establish a driving role for ICA69 in the development of pSS.

ICA69 Autoimmunity in Primary SS Patients

To determine if ICA69 was an autoimmune target in patients with primary SS, we first measured T cell responses to ICA69 and Tep69 in PBMC from patients with primary SS (n=9), systemic lupus erythematosus (SLE, n=6) and age-matched healthy controls (n=12) (FIG. 7A). Positive responses to both ICA69 and Tep69 were observed in 8 of 9 patients with primary SS and were absent in patients with SLE and in healthy controls (P 0.008 vs. SLE; P 0.004 vs. healthy controls). These data identify T cell autoimmunity to ICA69 as a common characteristic of primary SS in humans. The absence of ICA69/Tep69 specific T cell responses in SLE patients suggests that autoimmunity to ICA69 may be used as a marker to differentiate between the two diseases, which share several autoimmune targets.

Immunoblotting was employed with patient sera to detect the presence of autoantibodies against ICA69 (FIG. 7B). Consistent with the presence of anti-ICA69 T cell autoimmunity, sera from 8 of 9 pSS patients were positive for ICA69 antibodies. No immunoreactivity was observed in sera from SLE patients (n=6) or healthy controls (n=12) (FIG. 7B). Our data therefore establish ICA69 as an autoantigen in both NOD mouse and human pSS. The generation of more patient data and family studies are underway to determine the diagnostic significance of anti-ICA69 immunoreactivity in this disease.

In conclusion, the instant invention evidences a dramatic protection from pSS in ICA69-deficient NOD mice. The reduction of sialoadenitis in ICA69^{-/-} mice is most likely the result of absent ICA69-specific autoimmunity. This conclusion is supported by the presence of ICA69-specific T cell responses in submandibular lymph nodes and spleens of wild type NOD mice and in peripheral blood of patients with primary SS. These T cell proliferative responses and in particular the tight correlations between ICA69-specific autoimmunity and disease status during peptide-based immunotherapy further emphasizes the link between pSS and ICA69. While we can not completely rule out a role for functional properties of ICA69 in disease development, as the function of the molecule remains unclear, nevertheless, the identification of ICA69 as a new autoantigen in pSS may provide a new marker for disease diagnosis and a new target for disease preventive therapy.

It has been previously observed that NOD tolerance induction and disease protection by ABBOS are dose dependent peptide effects, with failure of tolerization and disease acceleration/precipitation at suboptimal peptide doses²¹. The observed variation of ABBOS effects on tolerization and pSS disease progression likely reflects variances in the rate of peptide release from the oily emulsion applied and/or subtle differences in T cell repertoires. There was no quantitative relationship between the extent of tissue lesions and T- or B cell autoimmunity in established pSS of patients and NOD mice, suggesting that these autoreactivities reflect more the presence than the extent of tissue damage. However, following immunotherapy, tissue infiltration and autoreactivity changed closely in parallel. Immunotherapy-induced changes in ICA69 autoimmune status may provide a read-out of effectiveness.

The search for autoantigens in pSS identified several members of nuclear complexes (e.g. SS-A/Ro, SS-B/La, and SS-56), as well as more tissue-specific antigens such as a-fodrin, b-fodrin, and the muscarinic M3 receptor¹². In addition to the submandibular and lacrimal glands, ICA69 is also expressed in pancreatic beta cells and nervous system tissue. A high incidence of up to 40% of pSS patients manifest neurological complications, often with polyneuropathy and the appearance of anti-neuronal autoantibodies²⁵⁻²⁷. It is conceivable that autoimmune targeting of ICA69 may play a role in spreading of autoimmune disease to nervous system tissue. This cytosolic molecule is a prominent target in human and NOD mouse pSS, type 1 diabetes and in MS, where different epitopes are targeted¹⁹. Studies are under way to determine if the shared targeting of Tep69/ABBOS is related to the high prevalence of DR3 in pSS, which is shared with diabetes^{28,29}.

T cells are believed to drive the histopathological changes in pSS, yet the significance of T cell targeting of autoantigens identified previously is not known³⁰. However, immunity to a-fodrin, was shown to be critical for the development of salivary and lacrimal gland exocrinopathy in NFS/sld mice⁶. Mild sialoadenitis does develop in ICA69 deficient mice, but with a considerable decrease in rate of progression and severity. These observations suggest that T cell targeting of ICA69 may be more central to the progression phase of disease after it has been initiated, possibly through autoimmune targeting of other autoantigens such as a-fodrin³¹. A hierarchy of autoantigen targeting and antigen spreading from few to many has been proposed in several autoimmune conditions.

Autoimmunity to ICA69 appears to be essential for the development of NOD mouse dacryoadenitis, a disease related to, but distinct from sialoadenitis by several criteria. ICA69^{-/-} NOD males as old as one year failed to develop histological signs of dacryoadenitis. Differences in disease development between lacrimal and salivary gland infiltration are common. pSS patients can develop sialoadenitis with or without dacryoadenitis and vice versa¹. A requirement for autoimmune targeting of ICA69 in the manifestation of dacryoadenitis identifies ICA69 as a critical antigen in the initiation of this disease. It will be interesting to determine if the pSS-like disease of other mouse strains, such as MRL/1 pr, also involves autoimmune targeting of ICA69. What elements may contribute to the loss of tolerance to ICA69 and subsequent priming of T and B cells? One factor may lie in the extensive remodeling and apoptosis observed in the salivary glands of NOD and immunodeficient NOD.scid mice^{32,33}. This process could liberate ICA69 antigen to draining lymph nodes or antigen presenting cells found in the tissue, subsequently resulting in T cell activation. Consistently, we observed spontaneous ICA69/Tep69-specific T cell responses in draining submandibular lymph nodes of 10 week old NOD females. General defects in the immune system likely contribute to disease. For example, elevated levels of the TNF superfamily member, B cell activating factor (BAFF), have been observed in pSS patients, and transgenic expression of BAFF produces pSS in C57BL/6 mice³⁴. Such abnormalities may promote systemic defects in self-tolerance, which may include prominent autoimmunity to ICA69.

Because of the diversity and variability of human pSS, translation of data from the NOD mouse to human disease must be met with caution. However, the identification of ICA69 as a novel and perhaps central autoantigen in pSS has ramifications. Antibodies to ICA69 could be used as markers in disease diagnosis and to serologically differentiate between pSS and SLE. In addition, non-toxic immunotherapies aimed at depleting ICA69/Tep69-reactive T cell pools could be a candidate therapy to halt and reverse disease progression.

In U.S. Pat. No. 6,207,389, the contents of which is incorporated herein in its entirety, we have previously associated the efficiency of ABBOS immunotherapy in diabetes prevention with its high affinity binding to MHC, where the tolerogenic effect of ABBOS was dose-dependent, and predictable disease exacerbation was observed at suboptimal doses²¹. This raises caution in the translation of mouse to human data, in particular with the choice of peptide and peptide doses. Thus, ABBOS homologs with even higher affinity should be considered for optimal and safer immunotherapy, which could be monitored with biopsies and T cell assays.

### REFERENCE RELIED UPON

1 Fox R I, Stern M, Michelson P. Update in Sjogren syndrome. Curr Opin Rheumatol 2000;12: 391-8.
2 James J A, Harley J B, Scofield R H. Role of viruses in systemic lupus erythematosus and Sjogren syndrome. Curr Opin Rheumatol 2001;13: 370-6.
3 Fox R I, Tornwall J, Michelson P. Current issues in the diagnosis and treatment of Sjogren's syndrome. Curr Opin Rheumatol 1999;11: 364-71.
4 Harley J B, Alexander E L, Bias W B, et al. Anti-Ro (SS-A) and anti-La (SS-B) in patients with Sjogren's syndrome. Arthritis Rheum 1986;29: 196-206.
5 Billaut-Mulot O, Cocude C, Kolesnitchenko V, et al. SS-56, a novel cellular target of autoantibody responses in Sjogren syndrome and systemic lupus erythematosus. J Clin Invest 2001;108: 861-9.
6 Haneji N, Nakamura T, Takio K, et al. Identification of alpha-fodrin as a candidate autoantigen in primary Sjogren's syndrome. Science 1997;276: 604-7.
7 Kuwana M, Okano T, Ogawa Y, Kaburaki J, Kawakami Y. Autoantibodies to the amino-terminal fragment of beta-fodrin expressed in glandular epithelial cells in patients with Sjogren's syndrome. J Immunol 2001;167: 5449-56.
8 Robinson C P, Brayer J, Yamachika S, et al. Transfer of human serum IgG to nonobese diabetic Igmu null mice reveals a role for autoantibodies in the loss of secretory function of exocrine tissues in Sjogren's syndrome. Proc Natl Acad Sci USA 1998;95: 7538-43.
9 Humphreys-Beher M G, Hu Y, Nakagawa Y, Wang P L, Purushotham K R. Utilization of the non-obese diabetic (NOD) mouse as an animal model for the study of secondary Sjogren's syndrome. Adv Exp Med Biol 1994;350: 631-6.
10 Hoffman R W, Alspaugh M A, Waggie K S, Durham J B, Walker S E. Sjogren's syndrome in MRL/l and MRL/n mice. Arthritis Rheum 1984;27: 157-65.
11 Haneji N, Hamano H, Yanagi K, Hayashi Y. A new animal model for primary Sjogren's syndrome in NFS/sld mutant mice. J Immunol 1994;153: 2769-77.
12 Brayer J B, Humphreys-Beher M G, Peck A B. Sjogren's syndrome: immunological response underlying the disease. Arch Immunol Ther Exp 2001;49: 353-60.
13 Robinson C P, Yamachika S, Bounous D I, et al. A novel NOD-derived murine model of primary Sjogren's syndrome. Arthritis Rheum 1998;41: 150-6.
14 Karges W, Hammond-McKibben D, Gaedigk R, Shibuya N, Cheung R, Dosch H M. Loss of self-tolerance to ICA69 in nonobese diabetic mice. Diabetes 1997;46: 1548-56.
Dosch H, Cheung R K, Karges W, Pietropaolo M, Becker D J. Persistent T cell anergy in human type 1 diabetes. J Immunol 9;163: 6933-40.
Pilon M, Peng X R, Spence A M, Plasterk R H, Dosch H M. The diabetes autoantigen ICA69 and its Caenorhabditis elegans homologue, ric-19, are conserved regulators of neuroendocrine secretion. Mol Biol Cell 2000;11: 3277-88.
Winer S, Astsaturov I, Gaedigk R, et al. ICA69(null) nonobese diabetic mice develop diabetes, but resist disease acceleration by cyclophosphamide. J Immunol 2002;168: 475-82.
18 Karges W, Pietropaolo M, Ackerley C, Dosch H M. Gene expression of islet cell antigen p69 (ICAp69) in man, mouse and rat. Diabetes 1996;45: 513-21.
19 Winer S, Astsaturov I, Cheung R K, et al. Type I Diabetes and MS Patients Target Islet plus CNS-Autoantigens, Non-immunized NOD Mice Can Develop Autoimmune Encephalitis. J Immunol 2001;166: 2832-41.
20 Serreze D V, Chapman H D, Varnum D S, et al. B lymphocytes are essential for the initiation of T cell-mediated autoimmune diabetes: analysis of a new “speed congenic” stock of NOD.Ig mu null mice. J Exp Med 1996;184: 2049-53.
21 Winer S, Gunaratnam L, Astsatourov I, et al. Peptide dose, MHC affinity, and target self-antigen expression are critical for effective immunotherapy of nonobese diabetic mouse prediabetes. J Immunol 2000;165: 4086-94.
22 Dosch H-M, Becker D J. Measurement of T-cell autoreactivity in autoimmune diabetes. Diabetologia 2000;43: 386-7.
23 Hunger R E, Carnaud C, Vogt I, Mueller C. Male gonadal environment paradoxically promotes dacryoadenitis in nonobese diabetic mice. J Clin Invest 1998;101: 1300-9.
24 Chen W, Bergerot I, Elliott J F, et al. Evidence that a peptide spanning the B-C junction of proinsulin is an early Autoantigen epitope in the pathogenesis of type 1 diabetes. J Immunol 2001;167: 4926-35.
25 Lafitte C, Amoura Z, Cacoub P, et al. Neurological complications of primary Sjogren's syndrome. J Neurol 2001;248: 577-84.
26 Andonopoulos A P, Lagos G, Drosos A A, Moutsopoulos H M. The spectrum of neurological involvement in Sjogren's syndrome. Br J Rheumatol 1990;29: 21-3.
27 Malinow K, Yannakakis G D, Glusman S M, et al. Subacute sensory neuronopathy secondary to dorsal root ganglionitis in primary Sjogren's syndrome. Ann Neurol 1986;20: 535-7.
Chused T M, Kassan S S, Opelz G, Moutsopoulos H M, Terasaki PI. Sjogren's syndrome association with HLA-Dw3. N Engl J Med 7;296: 895-7.
29 Foster H, Stephenson A, Walker D, Cavanagh G, Kelly C, Griffiths I. Linkage studies of HLA and primary Sjogren's syndrome in multicase families. Arthritis Rheum 1993;36: 473-84.
30 Goillot E, Mutin M, Touraine J L. Sialadenitis in nonobese diabetic mice: transfer into syngeneic healthy neonates by splenic T lymphocytes. Clin Immunol Immunopathol 1991;59: 462-73.
31 Yanagi K, Ishimaru N, Haneji N, Saegusa K, Saito I, Hayashi Y. Anti-120-kDa alpha-fodrin immune response with Th1-cytokine profile in the NOD mouse model of Sjogren's syndrome. Eur J Immunol 1998;28: 3336-45.
32 Masago R, Aiba-Masago S, Talal N, et al. Elevated proapoptotic Bax and caspase 3 activation in the NOD.scid model of Sjogren's syndrome. Arthritis Rheum 2001;44: 693-702.
33 Robinson C P, Yamamoto H, Peck A B, Humphreys-Beher M G. Genetically programmed development of salivary gland abnormalities in the NOD (nonobese diabetic)-scid mouse in the absence of detectable lymphocytic infiltration: a potential trigger for sialoadenitis of NOD mice. Clin Immunol Immunopathol 1996;79: 50-9.
34 Groom J, Kalled S L, Cutler A H, et al. Association of BAFF/BLyS overexpression and altered B cell differentiation with Sjogren's syndrome. J Clin Invest 2002;109: 59-68.

The above references were relied upon and are incorporated by reference herein in their entirety.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. An in vitro process for the differential diagnosis of primary Sjögren's Syndrome versus systemic lupus erthematosis in a blood sample comprising:
determining the presence therein of autoantibodies and/or T cell autoreactivity to ICA69;
whereby the presence of said autoimmune markers confirms a diagnosis of primary Sjögren's Syndrome.

2. Use of a specific peptide targeting ICA69-specific T cells, selected from the group consisting of:
i) an oligopeptide having the amino acid sequence SEQ. ID No. 1 (Tep69); and
ii) an oligopeptide having the amino acid sequence SEQ. ID No. 2 (ABBOS),
for alleviating and/or reversing the progression of primary Sjögren's Syndrome, whereby a reduction in the symptoms characteristic of primary Sjögren's Syndrome is attained.

3. Use according to claim 2, wherein said primary Sjögren's Syndrome is late stage primary Sjögren's Syndrome and whereby a reversal of sialoadenitis and dacryadenitis associated with late stage primary Sjögren's Syndrome is attained.

4. An in vitro assay for monitoring the disease status of a patient diagnosed with primary Sjögren's Syndrome comprising;
periodically analyzing said blood sample from said patient for the presence and or quantity of ICA69-specific T cells;
whereby the presence or relative increase or decrease in ICA69 specific T cells is indicative of changes in disease status of said patient.


SEQUENCE LISTING (2 sequences)

SEQ ID NO: 1 - protein, 12 aa, Artificial
  T-cell self-epitope in ICA69
AFIKATGKKEDE

SEQ ID NO: 2 - protein, 15 aa, Artificial
  T-cell epitope in bovine serum albumin
FKADEKKFWGKYLYE